(19) United States
(12) Patent Application Publication
(10) Pub. No.: US 2023/0049572 A1
(43) Pub. Date: Feb. 16, 2023

(54) FORMULATION, COMPOSITION OR FOODSTUFF ADDITIVES FOR THE MODIFICATION OF GLYCEMIC RESPONSE METHODS OF MANUFACTURING AND USING THE SAME

(71) Applicant: HOOW FOODS PTE. LTD., Singapore (SG)

(72) Inventor: Sherman Si Han HO, Singapore (SG)

(73) Assignee: HOOW FOODS PTE. LTD., Singapore (SG)

(21) Appl. No.: 17/786,290

(22) PCT Filed: Dec. 10, 2020
(86) PCT No.: PCT/SG2020/050739

§ 371 Date: Jun. 16, 2022

(30) Foreign Application Priority Data
Dec. 16, 2019 (SG) 10201912271S

(51) Int. Cl. A61K 31/7048; A61K 47/69; A61K 31/7008; A61K 31/7004; A61K 9/14; A61K 31/352; A61K 31/353; A61K 31/121; A61P 3/10; A23L 33/105; A23L 33/125

(52) U.S. Cl. CPC A61K 31/7048; A61K 47/6951; A61K 31/7008; A61K 31/7004; A61K 9/146; A61K 31/352; A61K 31/353; A61K 31/121; A61P 3/10; A23L 33/105; A23L 33/125; A23V 2002/00

(57) ABSTRACT

The present disclosure relates generally to formulations, compositions or foodstuff additives for use in modulating a glycemic response for treating or preventing diabetes or obesity and processes and method of its manufacture. Described is a formulation, a composition or a foodstuff for modulating a glycemic response manufactured from at least one phenylpropanoid encapsulated in a first cyclodextrin; and a second cyclodextrin. Preferably, the at least one phenylpropanoid is quercetin, phlorizin, myricetin, dihydromyricetin or any combination thereof, the first cyclodextrin is gamma cyclodextrin, and the second cyclodextrin is alpha cyclodextrin.

### CROSS REFERENCE TO RELATED APPLICATIONS

This application claims the priority to Singapore patent application No. 10201912271S, filed 16 Dec. 2019, the contents of which are incorporated herein by reference.

### FIELD

The present disclosure relates generally to formulations, compositions or foodstuff additives for use in modulating a glycemic response and method of manufacturing and using the same.

### BACKGROUND

The following discussion of the background to the invention is intended to facilitate an understanding of the present invention only. It should be appreciated that the discussion is not an acknowledgement or admission that any of the material referred to was published, known or part of the common general knowledge of the person skilled in the art in any jurisdiction as at the priority date of the invention.

Conventional foods are often high in sugar as well as refined flour, the latter of which presents highly digestible starch to the consumer's gut. Combined with a general problem of overeating especially in developed countries, these have contributed significantly to the increase in the incidence of diabetes worldwide. Singapore has one of the highest incidence rates of diabetes. There is a push for consumers to accept foods and practices which will not spike blood glucose as much. These include using brown sugar or honey in place of refined sugar, and wholegrains or wholemeal flour in place of plain flour. However, these are partial solutions, in the sense that they alter the taste and texture of the foods they were introduced to. This leads to a less than favourable uptake.

Diabetes Mellitus (DM), is a group of metabolic disorders characterised by high blood sugar levels over prolonged period of time. As of 2017, an estimated 425 million people had diabetes worldwide (Diabetes atlas 2017). Diabetes is due to either the pancreas not producing enough insulin, or the cells of the body not responding properly to the insulin produced. There are three main types of diabetes mellitus:
Type 1 diabetes results from the pancreas's failure to produce enough insulin due to loss of beta cells.
Type 2 diabetes begins with insulin resistance, a condition in which cells fail to respond to insulin properly. As the disease progresses, a lack of insulin may also develop.
Gestational diabetes is the third main form, and occurs when a pregnant woman without a previous history of diabetes develop high blood sugar levels.

There is broad consensus that when people with diabetes maintain tight glycemic control by keeping the glucose levels in their blood within normal ranges, that they experience fewer complications. All forms of diabetes increase the risk of long-term complications. Currently, there is no definitive management for hyperglycemia (high glucose) besides good control of blood sugar level via a strict diet, regular monitoring and in many cases insulin injections when required. Managing blood sugar levels can be very difficult for some patients especially those who have to be on insulin injections, due to cost, and pain of injections and monitoring. It is also difficult for people to maintain a strict diet. There is a need to manage the glycemic response in a simple way that makes it less difficult for patients that fail to comply with regular monitoring, diet, or injections.

Another group of at-risk individuals are those with pre-diabetes that include factors that may lead to the onset of diabetes such as being obese, overweight, leading sedentary and unhealthy lifestyles. For this group they may rarely measure any blood glucose levels. Their medical practitioner may still wish to modulate the glycemic response of such individual for weight management and to reduce the risk of the onset of diabetes. It may be difficult to change the exercise habits and diets of such individuals. There is a need to manage the glycemic response to avoid weight gain and facilitate weight loss in individuals.

Compositions such as those mostly consisting of a mixture of fibres and gums that can dampen the glycemic response after eating have been described. Such compositions that rely largely on the presence of water absorbing fibres (soluble or insoluble) and gums/hydrocolloids tend to affect the texture of foods that they are added to. The texture of food is one of the primary attributes affecting its quality. Along with taste and smell, texture defines a food and how we perceive that food's flavour and mouth feel. Having a texture that we perceive as appropriate for the foodstuff concerned is vitally important to our enjoyment of food. Where there is a need for more healthful eating, food texture and the development of creative texture solutions are important.

Cyclodextrins are sometimes used as fibre replacements. Fibres, gums or fibre replacements are mechanistically simple, in the sense that fibre-based compositions aim to delay gastric emptying and slow down digestion to dampen the glycemic response.

Others have used mulberry leaf extract to reduce glycemic response through enzyme inhibition, primarily by 1-deoxynojirimycin (1-DNJ) which is standardized at either 1% or 5%. The same has been shown to have an upper efficacy limit of 250mg per dose above which no further reduction in glycemic response is obtained. Alternative enzymatic inhibition of digestion have been reported.

There have been many studies reporting the health benefits provided by phenylpropanoid such as bioflavonoids isolated from various plants. Some flavonols are reported to be digestive enzyme inhibitors, however, there are no products on the market that include phenylpropanoids for reducing the glycemic response, possibly as phenylpropanoids are considered unpalatable as they taste bitter and astringent. Some phenylpropanoids are also acid-sensitive. Further, phenylpropanoids are a relatively expensive ingredient.

There exists a need to have formulations compositions or foodstuff additives that alleviates at least one of the aforementioned problems.

### SUMMARY

A formulation, composition or foodstuff additive for modulating a glycemic response and methods of manufacturing the same that do not affect the texture or taste of the foodstuff to which it is added is envisaged.

Accordingly, an aspect of the invention refers to a formulation for modulating a glycemic response comprising: (a) at least two different phenylpropanoids encapsulated in a first cyclodextrin; (b) an iminosugar; (c) a monosaccharide-based enzyme inhibitor and (d) a second cyclodextrin.

Another aspect of the invention refers to a composition for modulating a glycemic response comprising: (a) at least one phenylpropanoid encapsulated in a first cyclodextrin; and (b) a second cyclodextrin.

According to another aspect there is a foodstuff additive including the formulation as described herein above or the composition as described herein above.

According to another aspect there is a formulation as described herein above; a composition as described herein above, or the foodstuff additive as described herein above, for use in the treatment or prevention of diabetes or obesity.

According to another aspect there is a method of manufacturing a formulation as described herein above; a composition as described herein above; or the foodstuff additive as described herein above, for use in the treatment or prevention of diabetes or obesity.

According to another aspect there is a process for manufacturing a formulation for modulating a glycemic response comprising: (a) mixing at least two different phenylpropanoids and a first cyclodextrin; (b) adding water to the mix of at least two different phenylpropanoids and the first cyclodextrin to form a paste; (c) kneading the paste with shear force; (d) drying the paste; (e) grinding the dried paste to a powder; and (f) adding an iminosugar, a monosaccharide-based enzyme inhibitor and a second cyclodextrin to the powder to constitute the formulation, wherein the powder comprises the phenylpropanoids encapsulated in the first cyclodextrin.

According to another aspect there is a method of manufacturing a composition for modulating a glycemic response comprising: (a) mixing at least one phenylpropanoid and a first cyclodextrin; (b) adding water to the mix of at least one phenylpropanoid and the first cyclodextrin to form a paste; (c) kneading the paste with shear force; (d) drying the paste; (e) grinding the dried paste to a powder; and (f) adding a second cyclodextrin to the powder to form the composition, wherein the powder comprises the at least one phenylpropanoid encapsulated in the first cyclodextrin.

According to another aspect there is a method for treating or preventing diabetes comprising administering to an individual and amount of the formulation as described herein above or the composition as described herein above, or the foodstuff additive as described herein above to reduce the glycemic response of the individual.

According to another aspect there is a method for treating or preventing obesity comprising administering to an individual and amount of the composition described herein above, or the foodstuff additive described herein above to reduce the glycemic response, slow down digestion and/or maintain post-prandial satiety of the individual.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of non-limiting examples only, embodiments of the present invention,

FIG. 1: A schematic for manufacturing a formulation.

FIG. 2: Absolute blood glucose levels from all subjects, control and formulation 1 test.

FIG. 3: Average blood glucose levels with standard deviation, control and formulation 1 test.

FIG. 4: Difference from baseline for all subjects, control and formulation 1 test.

FIG. 5: Average difference from baseline with standard deviation, control and formulation 1 test.

FIG. 6: Depictions of foods made with (A): 10% formulation 2 in flour; (B): normal flour; (C & D) 6% formulation 2 in flour; (E&F) 5% of formulation 2 in rice flour.

FIG. 7: Average blood glucose levels with standard deviation, control and formulation 2 test.

FIG. 8: Glycemic index response curves for (A) reference food (glucose); test food—white bread made with 6% formulation 2 in flour (white bread LD) n=9; and (B) reference food (glucose); test food—plain unadulterated white bread (white bread) n=12.

### DETAILED DESCRIPTION

Throughout this document, unless otherwise indicated to the contrary, the terms “comprising”, “consisting of”, “having” and the like, are to be construed as non-exhaustive, or in other words, as meaning “including, but not limited to”.

Furthermore, throughout the document, unless the context requires otherwise, the word “include” or variations such as “includes” or “including” will be understood to imply the inclusion of a stated integer or group of integers but not the exclusion of any other integer or group of integers.

Unless defined otherwise, all other technical and scientific terms used herein have the same meaning as is commonly understood by a skilled person to which the subject matter herein belongs.

According to an aspect of the invention there is a formulation for modulating a glycemic response comprising: (a) at least two different phenylpropanoids encapsulated in a first cyclodextrin; (b) an iminosugar; (c) a monosaccharide-based enzyme inhibitor and (d) a second cyclodextrin.

The formulation has multiple mechanisms of action that may be exploited in a single product to dampen the glycaemic response significantly. In some cases an unprecedented drop in glycemic index of 97.63% was observed. It is surmised that the formulation would also fulfil more functions than just dampening of glycemic index. As it is able to effectively slow down digestion through multiple mechanisms, such as being able to maintain post-prandial satiety for a longer period of time, which will be useful for weight management as it may lead to lower caloric intake. As phenylpropanoids are also antioxidants, there could be effects related to anti-aging, anti-inflammation and modulation of immunity.

As used herein the term ‘modulating a glycemic response’ refers to lowering reducing or dampening the glycemic response such as by a reduction of the area under curve of blood glucose levels after a meal, or decreasing the peak blood glucose level, or delaying the time point at which the peak is found. The composition is able to lower the glycemic response of the body to foods to which it is added. From another perspective, this may be regarded as lowering the glycemic index of the food.

According to another aspect of the invention there is a composition for modulating a glycemic response comprising: (a) at least one phenylpropanoid encapsulated in a first cyclodextrin; and (b) a second cyclodextrin.

The formulation or the composition has the advantage that the unpleasant taste of any phenylpropanoid used is hidden from the sensory receptors by encapsulating them within the cavity of the first cyclodextrin. The resulting complexes comprising at least one phenylpropanoid encapsulated in a first cyclodextrin, have no or little taste and are much more acceptable to the individual consuming the composition. This will have the added advantage that the phenylpropanoids will be protected from the acidic environment of the stomach allowing more of the at least one phenylpropanoid to reach the intestines. This also allows less phenylpropanoid to be used per composition reducing the cost of manufacture. Adding a second cyclodextrin that has not been complexed makes the composition behave similarly to known sugars and starches thereby minimising any effect on the texture of the foods to which it is added.

The phenylpropanoids are a diverse family of organic compounds that are synthesized by plants from the amino acids phenylalanine and tyrosine. Their name is derived from the six-carbon, aromatic phenyl group and the three-carbon propene tail of coumaric acid, which is the central intermediate in phenylpropanoid biosynthesis. In various embodiments, the at least one phenylpropanoid comprises at two or more phenylpropanoids. In various embodiments, the at least one phenylpropanoid comprises at least two phenylpropanoids or three or more phenylpropanoids. In various embodiments, the at least one phenylpropanoid comprises any one of 1, 2, 3, 4, 5 or 10 different phenylpropanoid/s. In various embodiments, the three phenylpropanoids or the at least two or the at least one phenylpropanoid comprises chalcones, stilbenes, aurones, flavonoids, or their associated C-, N-, or O-glycosides and their respective reduced or oxidized forms. In various embodiments, the three phenylpropanoids or the at least two or the at least one phenylpropanoid comprises quercetin, myricetin, luteolin, baicalein, baicalin, apigenin, kaempferol, dihydrochalcone glycoside phlorizin or any combination thereof. In various embodiments, the at least one flavonoid comprises: quercetin, myricetin, dihydromyricetin, luteolin, baicalein, apigenin, kaempferol, or any combination thereof. In various embodiments, the three phenylpropanoids or the at least two or the at least one phenylpropanoid comprises a flavonoid, a chalcone, or any combination thereof. In various embodiments, the three phenylpropanoids or the at least two or the at least one phenylpropanoid is selected from the group consisting of at least flavonoid, at least chalcone, and any combination thereof. In various embodiments, the at least one flavonoid comprises an anthoxanthin. In various embodiments, the at least one flavonoid comprises a flavonol. In various embodiments, the at least one flavonol comprises; quercetin, myricetin, dihydromyricetin, luteolin, baicalein, apigenin, kaempferol, or any combination thereof. In various embodiments, the at least one chalcone comprises: dihydrochalcone glycoside, or phloretin. In various embodiments, the at least one phenylpropanoid comprises two flavonols and one chalcone. In various embodiments, the three phenylpropanoids or the at least two or the at least one phenylpropanoid is selected from the group consisting of quercetin, myricetin, dihydromyricetin, dihydrochalcone glycoside, and any combination thereof. In various embodiments dihydrochalcone glycoside comprises phlorizin. In various embodiments, the three phenylpropanoids or the at least two or the at least one phenylpropanoid comprises quercetin, myricetin, and phlorizin.

In various embodiments quercetin comprises 2-(3,4-dihydroxyphenyl)-3,5,7-trihydroxy-4H-chromen-4-one. In various embodiments, quercetin has the following structure:

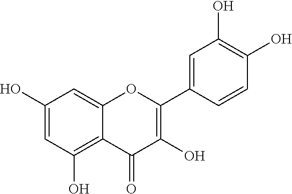 [SMILES: O=c1c(O)c(-c2ccc(O)c(O)c2)oc2cc(O)cc(O)c12]

In various embodiments myricetin comprises 3,5,7-Trihydroxy-2-(3,4 trihydroxyphenyl)-4-chromenone. In various embodiments, myricetin has the following structure:

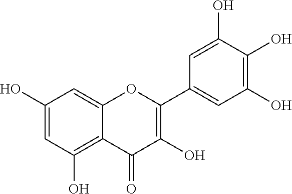 [SMILES: O=c1c(O)c(-c2cc(O)c(O)c(O)c2)oc2cc(O)cc(O)c12]

In various embodiments dihydromyricetin, (also referred to as Amelopsin or DHM) comprises (2R,3R)-3,5,7-Trihydroxy-2-(3,4,5-trihydroxyphenyl)-2,3-dihydrochromen-4-one. In various embodiments, dihydromyricetin has the following structure:

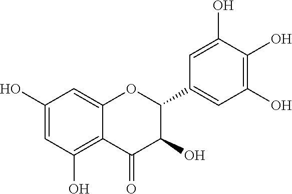 [SMILES: O=C1c2c(O)cc(O)cc2O[C@H](c2cc(O)c(O)c(O)c2)[C@H]1O]

In various embodiments phlorizin (also referred to as phloridzin) comprises phloretin-2′-β-D-glucopyranoside. In various embodiments, phlorizin has the following structure:

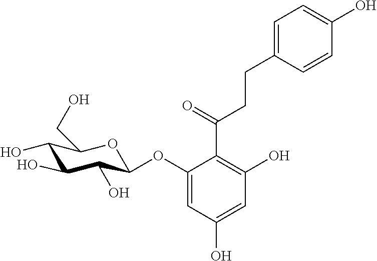

In the various embodiments where quercetin and myricetin are used in combination they advantageously have a synergistic activity. Individually, quercetin and myricetin inhibit amylase as well as alpha-glucosidases. When used together the inhibition is enhanced. In physiological context, this may translate to an inhibition of pancreatic amylase, digestive amylase, and enzymes sucrase-isomaltase and maltase-glycoamylase at the brush border of the small intestines. In the various embodiments where quercetin and myricetin are used in combination together with phlorizin, that possesses glucose transporter inhibition ability, specifically inhibiting Sodium glucose transporter 2 (SGLT2) and glucose transporter 2 (GLUT-2), the phenylpropanoid mix may be able to tackle the two aspects of glycemic response/index modulation, which is the inhibition of digestion of carbohydrates as well as inhibition of glucose uptake at the brush border of the small intestines.

Surprisingly, dihydromyricetin is very effective at inhibiting amylase as well as alpha-glucosidases. In the various embodiments dihydromyricetin used in combination together with phlorizin, that possesses glucose transporter inhibition ability, specifically inhibiting Sodium glucose transporter 2 (SGLT2) and glucose transporter 2 (GLUT-2), the phenylpropanoid mix may be able to tackle the two aspects of glycemic response/index modulation, which is the inhibition of digestion of carbohydrates as well as inhibition of glucose uptake at the brush border of the small intestines.

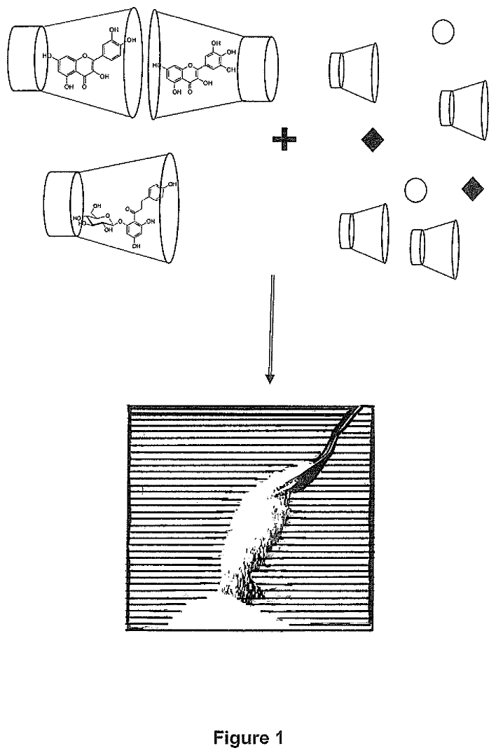

Cyclodextrins are oligosaccharides of glucopyranose units linked at alpha 1, 4 glycosidic bonds to form a ring. Because of the lack of free rotation around the bonds connecting the glucopyranose units, cyclodextrins are generally toroidal or cone shaped forming a hydrophobic central cavity (see FIG. 1). Various compounds may be able to be encapsulated within the hydrophobic central cavity. Adding a second cyclodextrin that has not been complexed makes the formulation or the composition behave in a similar way to other starch based products thereby minimising any effect on the texture of the foods to which it is added. In various embodiments, the first and second cyclodextrin may be different from the first cyclodextrin. In various other embodiments, the first and second cyclodextrin may be the same. In various embodiments, the first and second cyclodextrin are selected from the group consisting of alpha cyclodextrin, beta cyclodextrin and gamma cyclodextrin. In various embodiments, the first cyclodextrin comprises gamma cyclodextrin. In such embodiments the at least one phenylpropanoid may be released more rapidly, as gamma cyclodextrin is able to be partially hydrolysed by digestive amylases this allows for release of the phenylpropanoids in the intestine where they are meant to act. Further, gamma cyclodextrins are larger and is potentially able to accept at least two phenylpropanoids in its cavity. In various embodiments the second cyclodextrin comprises alpha cyclodextrin or beta cyclodextrin. Advantageously, both alpha cyclodextrin and beta cyclodextrin cannot be hydrolysed by pancreatic or salivary amylases. In various embodiments the second cyclodextrin comprises alpha cyclodextrin. In various embodiments, the first cyclodextrin comprises gamma cyclodextrin and the second cyclodextrin comprises alpha cyclodextrin. In such embodiments the alpha-cyclodextrin acts as an inhibitor of amylase, and also as fibre to slow gastric emptying.

In various embodiments, alpha cyclodextrin has 6 glucopyranose units having the following structure:

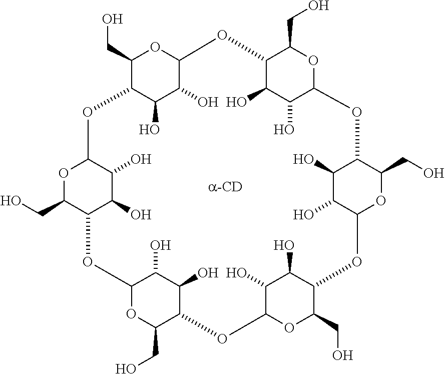

In various embodiments, beta cyclodextrin has 7 glucopyranose units having the following structure:

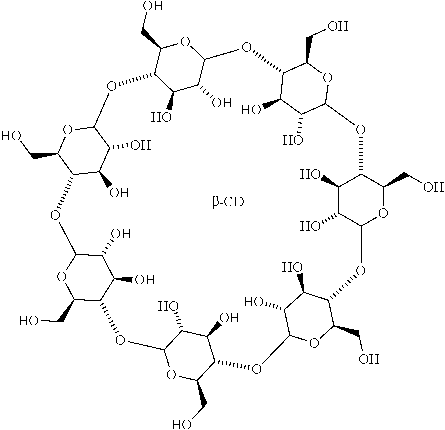

In various embodiments, gamma cyclodextrin has 8 glucopyranose units having the following structure:

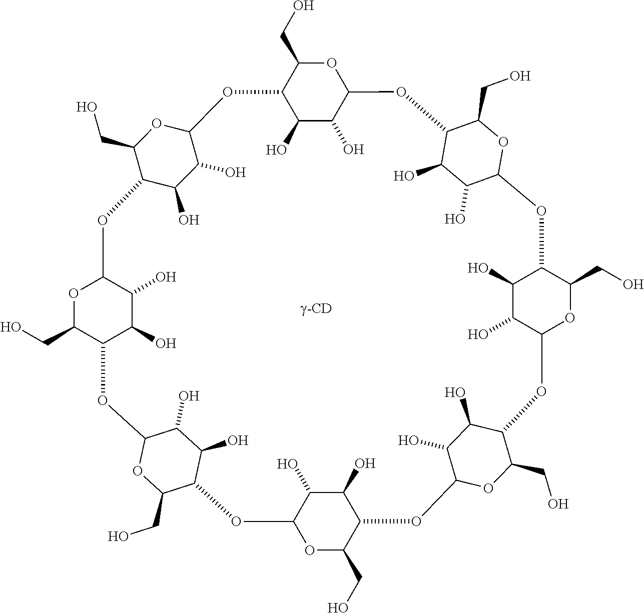

The volume of the central cavity differs for different cyclodextrins for example alpha cyclodextrins have a central cavity of about 0.10 ml/g; beta cyclodextrins have a central cavity of about 0.14 ml/g; and gamma cyclodextrins have a central cavity of about 0.20 ml/g. The larger the central cavity the more distance there is between the hydrophobic charges. It is generally difficult to include compounds in the larger sized cyclodextrins with 8 or more glucopyranose units such as gamma cyclodextrin. In various embodiments complexes of a mixture of two or more phenylpropanoid encapsulated in a gamma cyclodextrin was surprisingly possible. In such embodiments multiple mechanisms of action may be exploited in a single composition to dampen the glycemic response significantly.

It is surmised that the composition of such embodiments would also fulfil more functions than just dampening of glycemic index. As it is able to effectively slow down digestion through multiple mechanisms, such as being able to maintain post-prandial satiety for a longer period of time, which will be useful for weight management as it may lead to lower caloric intake. As phenylpropanoids are also well known to be antioxidants, there could be effects related to anti-aging, anti-inflammation and modulation of immunity.

In various embodiments the formulation or the composition advantageously involves a holistic approach to the dampening of the glycemic response and therefore reducing the glycemic index of foods “modulated” by the invention. There are three aspects that determine glycemic response of the body to foods. First, gastric emptying controls the rate at which partially digested food is presented to the digestive enzymes in the small intestine, which is where starch and sucrose are broken down to glucose and other monosaccharides for absorption through the small intestinal lumen. A meal rich in fat or high in fibre will retard gastric emptying, thereby slowing down digestion and dampening the glycemic response. Second, the presence and activity of digestive enzymes also determine the rate at which glucose is liberated from complex carbohydrates. The presence of inhibitors will slow this down as well, once again exerting a dampening effect on the glycemic response. Third, the presence and activity of glucose transporters on the intestinal lumen (brush border) also affects the rate at which the liberated glucose is absorbed and presented to the bloodstream. The first and second cyclodextrin retard gastric emptying, thereby slowing down digestion and dampening the glycemic response and the at least one phenylpropanoid encapsulated in the first cyclodextrin inhibit the digestive enzymes and glucose transporters at the site they are released.

In various embodiments, the composition further comprising iminosugar. In various embodiments the preferred iminosugar comprises 1-deoxynojirimycin (1-DNJ). In various embodiments the iminosugar comprises mulberry leaf extract where about 5% of the extract comprises is 1-DNJ. In various embodiments the iminosugar comprises 1-DNJ obtained from a modified microorganism able to produce 1 -DNJ. 1-DNJ is an iminosugar that inhibits amylase, alpha-glucosidases and possibly even glucose transporter. Co-administration with the complex described herein above, enhances the activity of this iminosugar that is known to reach a saturation point at a relatively low dose of 250 mg per meal. Used in combination both having broad spectrum inhibitory activity on the digestive enzymes and glucose transporters may provide an effective composition. In various embodiments pure 1-DNJ may be used, in various other embodiments 5% 1-DNJ may be used.

In various embodiments, the composition further comprises a monosaccharide-based enzyme inhibitor. In various embodiments the monosaccharide-based enzyme inhibitor comprises arabinose which is an inhibitor of sucrase-isomaltase. In various embodiments the monosaccharide-based enzyme inhibitor comprises sugars such as xylose, allulose or tagatose.

In various embodiments, the composition further comprises at least one free phenylpropanoid not encapsulated in any cyclodextrin. The at least one free phenylpropanoid may be any of the phenylpropanoid described herein above prior to encapsulation in a cyclodextrin.

In various embodiments, the first cyclodextrin of the formulation is gamma cyclodextrin; the at least two or the three different phenylpropanoids of the formulation are quercetin, myricetin, phlorizin; the iminosugar of the formulation comprises 1-deoxynojirimycin; the monosaccharide-based enzyme inhibitor of the formulation is arabinose; and the second cyclodextrin of the formulation is alpha cyclodextrin.

In various embodiments, the formulation of: gamma cyclodextrin:quercetin:myricetin:phlorizin:1-deoxynojirimycin:arabinose is in a ratio of 40:9:9:5:5:10:22 ora ratio of 35:9:9:4:5:37:1.

In various embodiments, the first cyclodextrin of the formulation is gamma cyclodextrin; the at least two different phenylpropanoids of the formulation are dihydromyricetin and phlorizin; the iminosugar of the formulation comprises 1-deoxynojirimycin; the monosaccharide-based enzyme inhibitor of the formulation is arabinose; and the second cyclodextrin of the formulation is alpha cyclodextrin.

In various embodiments, the formulation of: gamma cyclodextrin:dihydromyricetin:phlorizin:1-deoxynojirimycin:arabinose is in a ratio of 5:2:1:3:1:30 to a ratio of 25:6:6:10:10:90. In various embodiments, the formulation of: gamma cyclodextrin:dihydromyricetin:phlorizin:1-deoxynojirimycin:arabinose is 30:8:4:15:10:128.

In various embodiments, the formulation further comprises a flavour or colour varying additive. In various embodiments, the flavour is a bitterness masking flavour. In various embodiments, the colour varying additive comprises titanium dioxide. Titanium dioxide acts as a colour modifier to reduce the yellow tone of the product.

According to another aspect there is a foodstuff additive including the formulation or the composition as described herein above.

In various embodiments foodstuff comprises anything that can be eaten or consumed by an animal including a human. In various embodiments foodstuff comprise consumables containing carbohydrate. In various embodiments foodstuff comprises cooked rice, bread, cooked noodles or pasta, potato fries, chips, crisps, biscuits, cookies, cakes, beverages, or sauces.

In various embodiments a powdered foodstuff additive may be added into other ingredients or into finished food products for the purpose of lowering the glycemic index of said food. In various embodiments the powdered foodstuff additive may be spray coated onto sugar. In various embodiments the powdered foodstuff additive may be granulated into larger sized particles. In various embodiments the powdered foodstuff additive may be dry blend with sugar or flour.

According to another aspect there is a formulation as described herein above; a composition as described herein above, or a foodstuff additive as described herein above, for use in the treatment or prevention of diabetes or obesity.

The terms “prevent, prevention or preventing”, as used herein refer to reducing or lessening the glycemic response in a subject to lose weight, maintain an acceptable weight or to prevent the onset of diabetes.

The terms “treat, treatment or treating”, as used herein refer to reducing or lessening the glycemic response in a subject to maintain tight glycemic control by keeping the glucose levels in their blood within normal ranges, that the subject experiences fewer complications.

According to another aspect there is a method of manufacturing a composition as described herein above, or the foodstuff additive as described herein above, for use in the treatment or prevention of diabetes or obesity.

According to another aspect there is a process for manufacturing a formulation for modulating a glycemic response comprising: (a) mixing at least two different phenylpropanoids and a first cyclodextrin; (b) adding water to the mix of at least two different phenylpropanoids and the first cyclodextrin to form a paste; (c) kneading the paste with shear force; (d) drying the paste; (e) grinding the dried paste to a powder; and (f) adding an iminosugar, a monosaccharide-based enzyme inhibitor and a second cyclodextrin to the powder to constitute the formulation, wherein the powder comprises the phenylpropanoids encapsulated in the first cyclodextrin.

In various embodiments, as depicted in FIG. 1 the at least two or the different phenylpropanoids are complexed in the first toroidal or cone shaped cyclodextrin such that the phenylpropanoids encapsulated in the central cavity of the first cyclodextrin then the iminosugar represented by a circle, a monosaccharide-based enzyme inhibitor, represented as a square and a second toroidal or cone shaped cyclodextrin are added resulting in the formulation for modulating a glycemic response.

In various embodiments, the first and second cyclodextrin used in the process are different.

In various embodiments, the first and second cyclodextrin used in the process are selected from the group consisting of alpha cyclodextrin, beta cyclodextrin and gamma cyclodextrin.

In various embodiments, the first cyclodextrin used in the process comprises gamma cyclodextrin

In various embodiments, the at least two different phenylpropanoids used in the process comprises a flavonoid, a chalcone, or any combination thereof.

In various embodiments, the at least two different phenylpropanoids used in the process are selected from the group consisting of one or more flavonoid, at least chalcone, and any combination thereof.

In various embodiments, the flavonoid used in the process comprises any one of: quercetin, myricetin, dihydromyricetin, luteolin, baicalein, baicalin, apigenin, kaempferol, or any combination thereof.

In various embodiments, the at least one chalcone used in the process comprises: dihydrochalcone glycoside, or phloretin.

In various embodiments, the first cyclodextrin used in the process is gamma cyclodextrin; the at least two different phenylpropanoids used in the process are quercetin, myricetin, and phlorizin; the iminosugar used in the process comprises 1-deoxynojirimycin; the monosaccharide-based enzyme inhibitor used in the process is arabinose; and the second cyclodextrin used in the process is alpha cyclodextrin.

In various embodiments, the formulation of gamma cyclodextrin:quercetin:myricetin:phlorizin:1-deoxynojirimycin:arabinose is manufactured in a ratio of 40:9:9:5:5:10:22 ora ratio of 35:9:9:4:5:37:1 to manufacture the formulation.

In various embodiments, the first cyclodextrin used in the process is gamma cyclodextrin; the at least two different phenylpropanoids used in the process are dihydromyricetin, and phlorizin; the iminosugar used in the process comprises 1-deoxynojirimycin; the monosaccharide-based enzyme inhibitor used in the process is arabinose; and the second cyclodextrin used in the process is alpha cyclodextrin.

In various embodiments, the formulation of gamma cyclodextrin:dihydromyricetin:phlorizin:1-deoxynojirimycin:arabinose is manufactured in a ratio of 5:2:1:3:1:30 to a ratio of 25:6:6:10:10:90. In various embodiments, the formulation of: gamma cyclodextrin:dihydromyricetin:phlorizin:1-deoxynojirimycin:arabinose is 30:8:4:15:10:128.

In various embodiments, the process further comprises adding a flavour or colour varying additive. In various embodiments, the flavour is a bitterness masking flavour. In various embodiments, the colour varying additive comprises titanium dioxide. Titanium dioxide acts as a colour modifier to reduce the yellow tone of the product.

According to another aspect there is a method of manufacturing a composition for modulating a glycemic response comprising: (a) mixing at least one phenylpropanoid and a first cyclodextrin; (b) adding water to the mix of at least one phenylpropanoid and the first cyclodextrin to form a paste; (c) kneading the paste with shear force; (d) drying the paste; (e) grinding the dried paste to a powder; and (f) adding a second cyclodextrin to the powder to form the composition, wherein the powder comprises the at least one phenylpropanoid encapsulated in the first cyclodextrin.

In various embodiments kneading the paste with shear force may be effected by means of a high shear cutter, granulator, planetary mixer, food processor, mortar and pestle or any equipment capable of generating strong shearing forces.

In various embodiments, the first cyclodextrin and the second cyclodextrin used in the method of manufacture are different. In various other embodiments, the first cyclodextrin and the second cyclodextrin used in the method of manufacture may be the same. In various embodiments, the first cyclodextrin and the second cyclodextrin are individually each selected from the group consisting of alpha cyclodextrin, beta cyclodextrin and gamma cyclodextrin. In various embodiments, the first cyclodextrin comprises gamma cyclodextrin. In various embodiments the second cyclodextrin used in the method of manufacture comprises alpha cyclodextrin or beta cyclodextrin. Advantageously, both alpha cyclodextrin and beta cyclodextrin cannot be hydrolysed by pancreatic or salivary amylases. In various embodiments the second cyclodextrin used in the method of manufacture comprises alpha cyclodextrin. In various embodiments, the first cyclodextrin comprises gamma cyclodextrin and the second cyclodextrin comprises alpha cyclodextrin. In such embodiments the alpha-cyclodextrin acts as an inhibitor of amylase, and also as a fibre to slow gastric emptying.

In various embodiments, the at least one phenylpropanoid used in the method of manufacture comprises a flavonoid, a chalcone, or any combination thereof. In various embodiments, the at least one phenylpropanoid used in the method of manufacture comprises two or more phenylpropanoids. In various embodiments, the at least one phenylpropanoid used in the method of manufacture comprises at least two or more phenylpropanoids. In various embodiments, the at least one phenylpropanoid used in the method of manufacture comprises any one of 1, 2, 3, 4, 5 or 10 different phenylpropanoid/s. In various embodiments, the at least one phenylpropanoid used in the method of manufacture comprises chalcones, stilbenes, aurones, flavonoids, or their associated C-, N-, or O-glycosides and their respective reduced or oxidized forms. In various embodiments, the at least one phenylpropanoid used in the method of manufacture comprises quercetin, myricetin, dihydromyricetin, luteolin, baicalein, baicalin, apigenin, kaempferol, dihydrochalcone glycoside, phloretin, phlorizin or any combination thereof. In various embodiments, the at least one flavonoid comprises: quercetin, myricetin, dihydromyricetin, luteolin, baicalein, apigenin, kaempferol, or any combination thereof. In various embodiments, the at least one phenylpropanoid used in the method of manufacture comprises a flavonoid, a chalcone, or any combination thereof. In various embodiments, the at least one flavonoid comprises an anthoxanthin. In various embodiments, the at least one flavonoid comprises at least one flavonol. In various embodiments, the at least one flavonol comprises;

quercetin, myricetin, dihydromyricetin, luteolin, baicalein, apigenin, kaempferol, or any combination thereof. In various embodiments, the at least one chalcone comprises: dihydrochalcone glycoside, phlorizin or phloretin. In various embodiments, the at least one phenylpropanoid used in the method of manufacture comprises two flavonols and one chalcone. In various embodiments, the at least one phenylpropanoid used in the method of manufacture is selected from the group consisting of quercetin, myricetin, dihydromyricetin, dihydrochalcone glycoside, and any combination thereof. In various embodiments dihydrochalcone glycoside comprises phlorizin. In various embodiments, the at least one phenylpropanoid used in the method of manufacture comprises quercetin, myricetin, and phlorizin.

In various embodiments, the at least one phenylpropanoid used in the method of manufacture is selected from a group consisting of at least one flavonoid, at least one chalcone, and any combination thereof. In various embodiments, the at least one flavonoid used in the method of manufacture comprises: quercetin, myricetin, dihydromyricetin, luteolin, baicalein, baicalin, apigenin, kaempferol, phlorizin or any combination thereof. In various embodiments, the at least one chalcone used in the method of manufacture comprises: dihydrochalcone glycoside, or phloretin. In various embodiments, dihydrochalcone glycoside used in the method of manufacture comprises phlorizin.

In various embodiments, the at least one phenylpropanoid used in the method of manufacture is selected from quercetin, myricetin, dihydrochalcone glycoside, and any combination thereof. In various embodiments, the at least one phenylpropanoid used in the method of manufacture comprises quercetin, myricetin, and phlorizin

In various embodiments, the method of manufacture, further comprises adding iminosugar to the composition. In various embodiments the iminosugar used in the method of manufacture comprises 1-deoxynojirimycin (1-DNJ). In various embodiments the iminosugar used in the method of manufacture comprises pure 1-DNJ. In various other embodiments the iminosugar used in the method of manufacture comprises 5%1-DNJ. In various other embodiments the iminosugar used in the method of manufacture comprises mulberry leaf extract standardised to contain 5% 1-DNJ.

In various embodiments, the method of manufacture, further comprises adding a monosaccharide inhibitor to the composition.

In various embodiments, the method of manufacture, further comprises adding at least one free phenylpropanoid not encapsulated in any cyclodextrin. The at least one free phenylpropanoid may be any of the phenylpropanoid described herein above prior to encapsulation in a cyclodextrin.

According to another aspect there is a method for treating or preventing diabetes comprising administering to an individual and amount of the composition described herein above, or the foodstuff additive described herein above to reduce the glycemic response of the individual.

Other possible uses relate to the long-term management of blood glucose as an adjunct to a typical pharmacotherapeutic regime for Type 2 diabetes mellitus. In this respect, it is anticipated that the unique formulation or composition will decrease insulin resistance, improve glucose utilization and also exert anti-inflammatory effects, all of which contribute to an improvement in diabetes mellitus.

According to another aspect there is a method for treating or preventing obesity comprising administering to an individual and amount of the composition described herein above, or the foodstuff additive described herein above to reduce the glycemic response, slow down digestion and/or maintain post-prandial satiety of the individual.

Other possible uses relate to the use for weight management as it may lead to lower caloric intake.

It should be further appreciated by the person skilled in the art that variations and combinations of features described above, not being alternatives or substitutes, may be combined to form yet further embodiments falling within the intended scope of the invention.

As would be understood by a person skilled in the art, each embodiment, may be used in combination with other embodiment or several embodiments.

### EXAMPLES

Preparation of a Complex of Phenylpropanoid Encapsulated in a Cyclodextrin.

To a dry blend of flavonoids and cyclodextrin (CD) in a molar ratio of between 1:1 to 1:3 (between 7-40 g of flavonoids to 20-60 g cyclodextrin), add water in a 1:2 mass ratio (flavonoid+cyclodextrin mix:water) and knead vigorously until a smooth paste is formed. Kneading may be effected by means of a high shear cutter, granulator, planetary mixer, food processor, mortar and pestle of any equipment capable of generating strong shearing forces. Once the viscosity progresses into a hard paste that is no longer manipulable, a second mass of water equivalent to the paste may be added to thin it down and continue kneading. This is repeated once more, resulting in a total kneading time of around 2 hours and a total mass of water added between 5 to 10 mass ratios to the flavonoid:cyclodextrin mix. The resulting paste is a smooth paste of ranging in colour from off-white, to yellow to greenish.

This paste is then dried in an oven at 35-40° C. until moisture content is 5% or lower. The dried cake is then ground into a fine powder. Resulting in at least one phenylpropanoid encapsulated in a cyclodextrin.

Complex 1

| Component | Mass (g) |
|---|---|
| Quercetin | 5-15 |
| Phlorizin | 2-10 |
| Myricetin | 5-15 |
| gammaCD | 20-60 |
| water | 160-1,000 |

Complex 2

| Component | Mass (g) |
|---|---|
| Quercetin | 10 |
| Phlorizin | 5 |
| Myricetin | 8 |
| gammaCD | 40 |
| water | 500 |

Complex 3

| Component | Mass (g) |
|---|---|
| Quercetin | 2.5-12.5 |
| Phlorizin | 2-10 |
| Myricetin | 2.5-12.5 |
| gammaCD | 20-60 |
| water | 135-950 |

Complex 4

| Component | % |
|---|---|
| Quercetin | 8 |
| Phlorizin | 5 |
| Myricetin | 7 |
| gammaCD | 30 |
| water | 400 |

Complex 5

| Component | Complex (g) |
|---|---|
| Quercetin | 7.25 |
| Phlorizin | 2.51 |
| Myricetin | 1.8 |
| gammaCD | 20.02 |
| water | 255 |

Complex 6

| Component | % |
|---|---|
| Dihydromyricetin | 4 |
| Phlorizin | 2 |
| gammaCD | 15 |
| water | 400 |

Quercetin, myricetin and phlorizin were weighed into a mortar, together with gamma-cyclodextrin (gammaCD). 25 ml of water was added and using the pestle, the mixture was triturated vigorously. Viscosity of the paste increased sharply after 2 min, signifying the start of complexation. Another 12.59 ml of water was added and triturated again. 5.84 ml of water was added after 21 min had elapsed. 6.30 ml of water was added again after the paste hardened again at 40 min. The paste was triturated for a total of 1 hr and 45 min to afford a smooth greenish paste. In one example the paste was scrapped onto a glass evaporating dish and dried at 37° C. for 48 hours in a drying oven with full airflow. The hardened mass was then ground into a fine powder in a stainless-steel mortar and dried again for 2 hours in the drying oven with full airflow. Yield was 30.00 g, 95%. This powder was then dry blended with the rest of the ingredients in a stainless-steel mortar to afford a dull, greenish yellow powder, 47.70 g.

Further refinement will focus on optimizing the production process, mainly pertaining to the encapsulation of phenylpropanoids in the gamma-cyclodextrin. Characterisation tests have been performed to check for encapsulation efficiency (data not shown).

Manufacture of the formulation or composition

The powered complex of phenylpropanoid encapsulated in cyclodextrin has other components such as iminosugars (as 5% mulberry leaf extract), monosaccharides, uncomplexed phenylpropanoids and cyclodextrins for example alpha cyclodextrin (alphaCD) added and blended together.

Composition 1

| Component | % |
|---|---|
| 5% 1-DNJ mulberry leaf extract | 2.5-10 |
| Complex 1 | 25-97 |
| Arabinose | 0-25 |
| alphaCD | 0.5-40 |
| | 100 |

| Component | % |
|---|---|
| 5% 1 -DNJ mulberry leaf extract | 5 |
| Complex 2 | 63 |
| Arabinose | 12 |
| alphaCD | 20 |
| | 100 |

Composition 3

| Component | % |
|---|---|
| 5% 1 -DNJ mulberry leaf extract | 2.5-10 |
| Complex 3 | 20-77.5 |
| Arabinose | 20-60 |
| alphaCD | 0.5-10 |
| | 100 |

Composition 4

| Component | % |
|---|---|
| 5% 1-DNJ mulberry leaf extract | 5 |
| Complex 4 | 50 |
| Arabinose | 40 |
| alphaCD | 5 |
| | 100 |

Composition 5

| Component | % |
|---|---|
| 5% 1-DNJ mulberry leaf extract | 3-10 |
| Complex 6 | 18-37 |
| Arabinose | 1-10 |
| alphaCD | 30-90 |
| flavour | 0.5-5 |
| | 100 |

formulation 1

| Component | % | Actual (g) |
|---|---|---|
| 5% 1-DNJ mulberry leaf | 5 | 2.39 |
| extract | | |
| Complex 5 | 63 | 30.07 |
| Arabinose | 12 | 3.81 |
| alphaCD | 20 | 11.43 |
| | 100 | 47.70 |

This affords a final powdered product which can be then further processed as part of flour, sugar or packaged.

The compounds are made into a fine powder, and used as follows:
Replacement of 2-10% flour for use in baking purposes for products such as cookies, cakes, bread, brownies, amongst others
Replacement of 2-10% flour for use in producing pasta or noodles
Replacement of 2-10% refined sugar, brown sugar or other sugar products
Supplied as a free-flowing powder in sachets, bags, or other containers for use in adding to foods during or after processing. Examples include
Addition of 1-10% by weight to a baking mixture, frying batter, sauce, raw rice, beverage mix to be co-processed/cooked together.
Addition of 1-10% by weight to a finished sauce, cooked rice, cooked soupy dishes and prepared beverages.
Addition of 1-10% by weight as a dusting powder on dry foods, such as biscuits, crackers, battered fried foods.

In some examples the composition may also be supplied as
Compressed hard tablets, chewable tablets
Capsules
Dissolved in a aqueous vehicle of 20-100 ml as a “shot”-type supplement drink
Single-dose sachets for dissolution into water or other beverage for taking every morning or as intended.

Testing the use of the Formulation 1

Preliminary In Vivo Investigations of Effect on Glycemic Response to White Bread with or without Modulation by a Novel Glycemic Index Modulator for Starchy Foods. Formulation 1 was produced on a lab scale (50 g). This was tested by ingestion of 10 g each on 4 subjects and showed positive in vivo results on the dampening of the glycemic response after eating 100 g of white bread.

A novel formulation or composition which is presumably able to dampen the glycemic response of foods taken together with it has been developed, hereafter termed “GI Mod 1” (formulation 1). It takes the form of a water dispersible/partially soluble off-white to green powder with a slightly cereal taste. The present study was designed to directly investigate the function of the GI Mod 1 (formulation 1) in vivo to determine its effects on the modulation of the glycemic response to a starchy food such as white bread.

Glycemic Index Study

4 male Chinese normo-glycemic subjects were recruited in this study with the following profiles:

TABLE 2

| Test subject profiles | | | |
|---|---|---|---|
| Identifier | Age | Height | Weight |
| SH | 33 | 182 cm | 86 kg |
| YP | 32 | 179 cm | 74 kg |
| HX | 36 | 169 cm | 83 kg |
| TY | 32 | 185 cm | 90 kg |

The study design is a paired-sample, controlled study. Specifically, each subject was studied on two consecutive days, and the study was started at lunch time between 12noon-1:30pm with a 3 hour fasting prior (water allowed). The study was conducted over the period of 4 Sep. 2019 to 10 Sep. 2019.

Control food sample was 100 g of sliced white bread (Gardenia Enriched White Bread), with 100-150 ml of plain water. 100 g sliced white bread contained about 57 g of carbohydrates. Test food sample consisted of the same, with an additional 10 g of the GI Mod 1 (formulation 1) in powder form, to be consumed together with the test food sample. GI Mod 1 (formulation 1) could be consumed by scattering between slices of bread, or dissolved into water and drunk. Subjects were given 10 min for consumption of the food samples.

Blood glucose levels in mmol/L were measured with a glucometer based on the glucose oxidase assay (Abbott Optium Neo). Blood samples were obtained with the supplied lancet and lancing device. Each sample was taken from a different spot on the fingers of both hands. A total of 7 samples were taken per subject per test, with the time points 0 (before consumption of test food), 15, 30, 45, 60, 90 and 120 minutes post consumption of test food.

Blood glucose levels were plotted against time, and the difference from baseline (0 min) was calculated and plotted as well. Incremental Area Under Curve (iAUC) was calculated and for the control sample, this was pegged to GI 100. The effect of GI Mod 1 (formulation 1) was therefore evaluated through the division of the iAUC (test) against iAUC (control) multiplied by 100.

Results

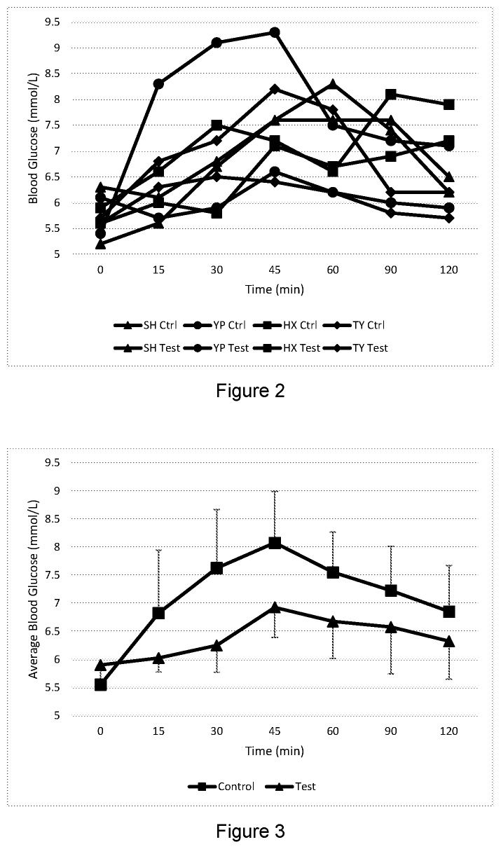

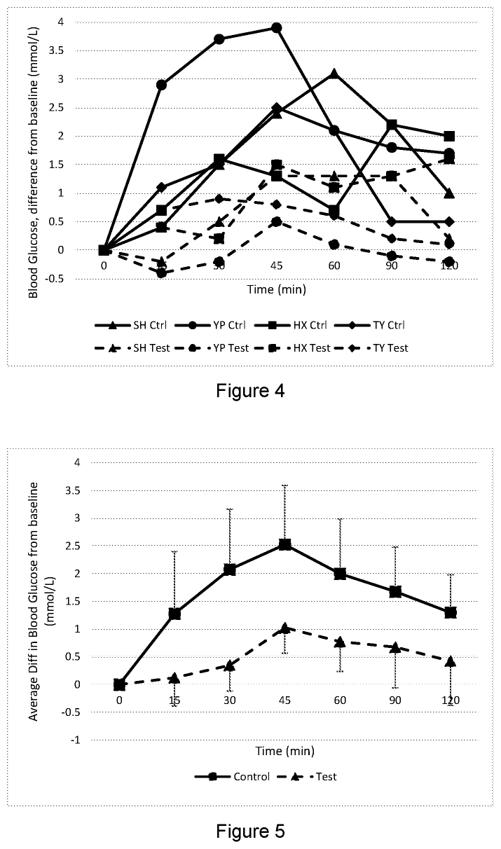

The absolute blood glucose graphs and difference from baseline graphs are shown in FIGS. 2 to 5

In summary, it is obvious that blood glucose levels taken when the individuals consumed the test composition are generally lower than the control, indicating a dampening effect on the glycemic response of the test subjects to the food sample. The time to peak concentration was not affected notwithstanding. Calculatina iAUCs for the above data produces the results below:

TABLE 3
iAUC values for control and test samples, and calculated Glycemic
Index of food sample

| Test | Control | Test | Control | | |
|---|---|---|---|---|---|
| subject | (mmol/L*min) | (mmol/L*min) | GI | Test GI | % change |
| SH | 212.25 | 96.75 | 100 | 44.95 | −55.05 |
| YP | 284.25 | 6.75 | 100 | 2.37 | −97.63 |
| HX | 165.75 | 119.25 | 100 | 71.95 | −28.05 |
| TY | 146.25 | 57.00 | 100 | 38.97 | −61.03 |
| Average | 221.75 | 74.25 | 100 | 33.48 | −66.52 |

All subjects experienced a significant dampening effect on the glycemic response to white bread, resulting in a 28.05-97.63% drop in glycemic index of the white bread. On average, the dampening effect on the GI was 66.52%, unprecedentedly strong in comparison to any known compositions. Astoundingly, the subject YP experienced a dampening effect so strong that it seemed that he had not consumed the bread at all. On the other hand, HX, which presented with a less common double peak blood glucose profile, showed the least effect at 28.05%.

Two questions on satiety were also posed to evaluate if GI Mod 1 (formulation 1) had any effects on the next meal, which may have implications on its use as a weight management product.

“How long did you start feeling hungry after consuming the test meal?”

| Test subject | Control | Test |
|---|---|---|
| SH | 3 h | 6 h |
| YP | 2 h | 8 h |
| HX | 3 h | 4 h |
| TY | 2 h | 4 h |

“What is the quantity of your next meal compared to a usual day?”

| Test subject | Control | Test |
|---|---|---|
| SH | Same | −50% |
| YP | Same | −75% |
| HX | Same | Same |
| TY | +25% | −25% |

The above answers fell roughly in line with the test results above. YP, who experienced the largest dampening effect, also experienced a long delay in hunger and the next meal was also diminished drastically in quantity. HX, who experienced the smallest dampening effect, generally did not feel any difference post-test. In summary, there is potential for compositions described herein to exert some degree of hunger management after consumption, and it is desirable to employ this in formulating it as a product for weight and appetite management.

GI Mod 1 (formulation 1) has shown strong dampening effects on the glycemic response to a typical carbohydrate-heavy meal of 100g white bread when added at a proportion of 10% in relevance to the meal. The prototype form of a powder was consumable, and subjective opinions were that it tasted “tea-like” or “cereal-like” with little complaints of bitterness, which also indicates the successful taste-masking of the phenylpropanoids by gamma cyclodextrin. Some extended satiety effect was also noted and no side effects were reported.

formulation 2

| Component | % |
|---|---|
| 5% 1-DNJ mulberry leaf extract | 4.5 |
| Complex 4 | 40.5 |
| Arabinose | 9 |
| alphaCD | 36 |
| Flavour (bitterness masking | 2.5 |
| agent) | |
| Titanium dioxide | 7.5 |
| | 100 |

Formulation 2 was made in a similar way to Formulation 1. The flavour is a bitterness masking agent added in this formulation the flavour was Smoothenol™ (N13917 from Sensient). Titanium dioxide was added to act as a colour modifier to reduce the yellow tone of the product. This is a typical function of titanium dioxide in food.

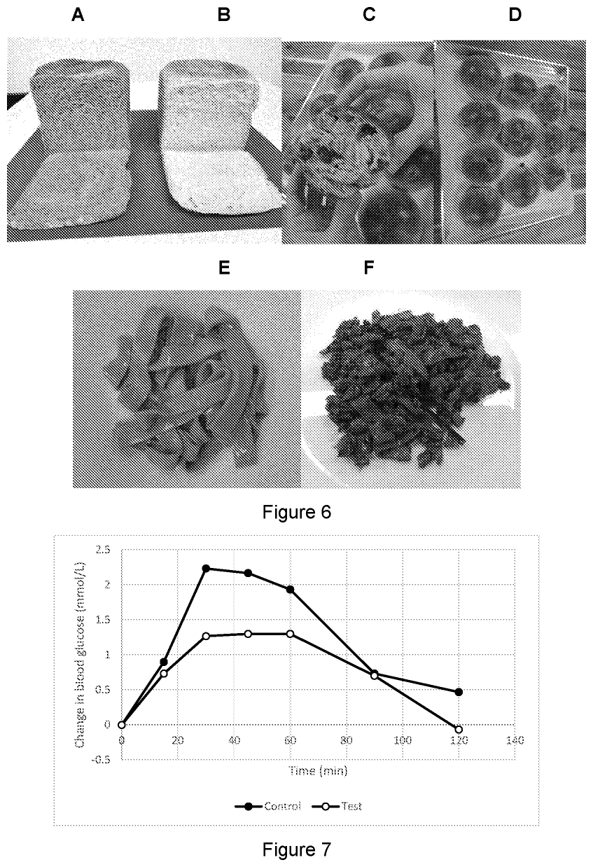

Flour with 10% of formulation 2 was baked into a Pullman loaf (FIG. 6A). Flour with 6% of formulation 2 was baked into a typical cranberry bun recipe (FIG. 6C-6D). Lastly, flour with 5% of formulation 2 was added to traditional Chinese flat rice noodles, kway teow (FIG. 6E). This kway teow could be fried into a Fried Kway Teow dish without any problems (FIG. 6F).

The control and test Pullman loaves above were used for an internal GI test according to the following:

3 male Chinese normo-glycemic subjects were recruited in this study with the following profiles:

| Identifier | Age | Height | Weight |
|---|---|---|---|
| SH | 33 | 182 cm | 86 kg |
| YP | 32 | 179 cm | 74 kg |
| TY | 32 | 185 cm | 90 kg |

Test Subject Profiles

The study design was a paired-sample, controlled study. Specifically, each subject was studied on two days, and the study was started at lunch time between 12noon-1:30 pm with a 3 hour fasting with water allowed prior to the test. The study was conducted over the period of 5 Mar. 2019 to 10 Sep. 2019.

Control food sample was 100g of sliced white bread, in-house Pullman loaf made in the same way as the test food sample without formulation 2. The control food sample was taken with 100-150 ml of plain water. 100g sliced white bread contained about 50 g of carbohydrates. The test food sample consisted of 100 g of sliced bread made with the 10% of formulation 2 in flour as described above. The test food sample was taken with 100-150 ml of plain water. Subjects were given 10 min for consumption of the food samples.

Blood glucose levels in mmol/L were measured with a glucometer based on the glucose oxidase assay (Abbott Optium Neo). Blood samples were obtained with the supplied lancet and lancing device. Each sample was taken from a different spot on the fingers of both hands. A total of 7 samples were taken per subject per test, with the time points 0 (before consumption of test food), 15, 30, 45, 60, 90 and 120 minutes post consumption of test food.

Blood glucose levels were plotted against time, and the difference from baseline (0 min) was calculated and plotted as well (FIG. 7). Incremental Area Under Curve (iAUC) was calculated and for the control sample, this was pegged to GI 100. The effect of GI Mod 1 (formulation 2) was therefore evaluated through the division of the iAUC (test) against iAUC (control) multiplied by 100.

Results

The absolute blood glucose graphs and difference from baseline graphs are shown in FIG. 7.

In summary, it is obvious that blood glucose levels taken when the individuals consumed the test composition are generally lower than the control, indicating a dampening effect on the glycemic response of the test subjects to the food sample. The time to peak concentration was not affected consistently notwithstanding. Calculating iAUCs for the above data produces the results below:

TABLE 4
iAUC values for control and test samples, and calculated
Glycemic Index of food sample

| | Control | Test | | | |
|---|---|---|---|---|---|
| Test | (mmol/L * | (mmol/L * | Control | | % |
| subject | min) | min) | GI | Test GI | change |
| SH | 177.75 | 149.25 | 100 | 83.97 | −16.03 |
| YP | 125.25 | 65.25 | 100 | 52.10 | −47.90 |
| TY | 160.50 | 81.75 | 100 | 50.94 | −49.07 |
| Average | 152 | 93.25 | 100 | 61.35 | −38.65 |

All subjects experienced a significant dampening effect on the glycemic response to white bread, resulting in a 16.03-49.07% drop in glycemic index of the white bread. On average, the dampening effect on the GI was 38.65%.

Two questions on satiety were also posed to evaluate if the above composition had any effects on the next meal, which may have implications on its use as a weight management product.

“How long did you start feeling hungry after consuming the test meal?”

| Test subject | Control | Test |
|---|---|---|
| SH | 2 h | 3 h |
| YP | 2 h | 6 h |
| TY | 2 h | 3 h |

“What is the quantity of your next meal compared to a usual day?”

| Test subject | Control | Test |
|---|---|---|
| SH | Same | Same |
| YP | Same | −75% |
| TY | −10% | −25% |

The above answers fell roughly in line with the test results above. YP, who experienced the second largest dampening effect, also experienced a long delay in hunger and the next meal was also diminished drastically in quantity. SH, who experienced the smallest dampening effect, generally did not feel any difference post-test. In summary, there is potential for compositions described herein to exert some degree of hunger management after consumption, and it is desirable to employ this in formulating it as a product for weight and appetite management.

Taste-wise, all three subjects mentioned that the texture of both breads were the same, and there were no bitter tastes compared to an earlier prototype.

Glycemic Index Study

The procedure was conducted at an ISO26642:2010 glycemic index testing facility.
Subjects were fasted for 8 hours
Subjects were provided either glucose solution (equivalent to 50 g glucose) or about 108 g of bread sample (equivalent to 50 g of net carbohydrates) to consume
Subjects had blood drawn at timepoints 0 (before eating), 15, 30, 45, 60, 90 and 120 minutes post eating and blood was assayed for glucose concentration.
iAUC were compared between the curves for bread and glucose to derive the glycemic index.

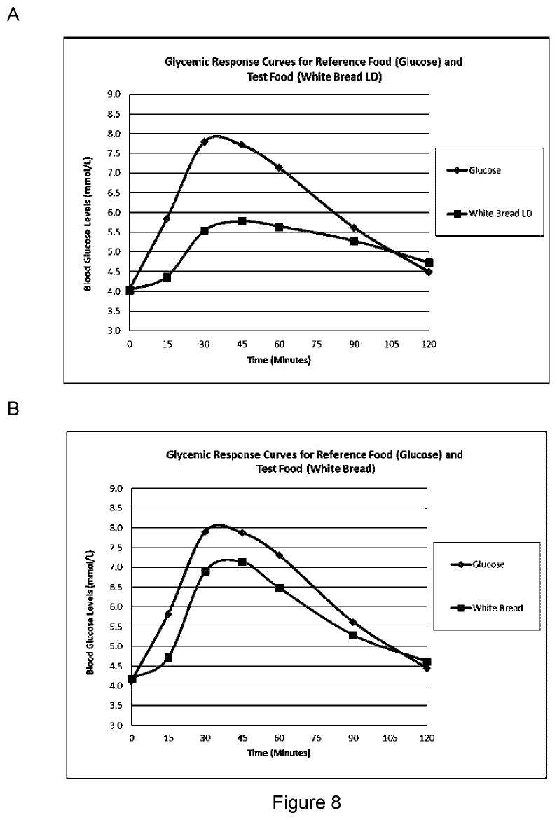

The peak glycemic index for plain unadulterated white bread (white bread) was 70 (FIG. 8B), whereas the peak glycemic index for white bread made with 6% formulation 2 in flour (white bread LD) was 55 (FIG. 8A). This represents a 21% reduction.

Formulation 3

| Component | % |
|---|---|
| 5% 1-DNJ mulberry leaf extract | 5 |
| Complex 4 | 30 |
| Arabinose | 40 |
| alphaCD | 22.5 |
| Flavour (bitterness masking agent) | 2.5 |
| | 100 |

Separately, the formulation 2 was revised to formulation 3 to reduce the glycemic index of sugar. This composition is amenable for typical fine grained or caster sugar, or sugars of varying degrees of refinement such as jaggery, brown, muscovado, turbinado, raw sugar etc. as it is high in Arabinose and Mulberry leaf extract which are natural inhibitors of sucrase enzyme.

Formulation 4

| Component | % |
|---|---|
| 5% 1-DNJ mulberry leaf extract | 7.5 |
| Complex 6 | 21 |
| Arabinose | 5 |
| alphaCD | 64 |
| Flavour (bitterness masking agent) | 2.5 |
| | 100 |

Formulation 4 was created for modulating the glycemic index of rice. This composition may be added into rice during the cooking process at a mass percentage of 3-10%.

Dihydromyricetin and phlorizin are first complexed with gamma-cyclodextrin to form complex 6, dried, ground to a powder and then combined with the other components of formulation 2.

The flavour or bitterness masking agent added in this formulation was Smoothenol™ (N 13917 from Sensient).

As dihydromyricetin is a white compound, the final cooked rice with the above composition retains the familiar white colour.

In Vitro Testing of Flavonoid Inhibition of Alpha-Glucosidase

The alpha-glucosidase inhibition was determined using purified alpha-glucosidase from yeast. An alpha-glucosidase reaction mixture contained 2.9 mM p-nitrophenyl-α-d-glucopyranoside (pNPG) (Sigma-Aldrich), 0.25 ml of flavonoid in DMSO and 0.6 U/ml of bakers yeast a-glucosidase (Sigma-Aldrich) in sodium phosphate buffer, pH 6.9. Control tubes contained only DMSO, enzyme and substrate, while in positive controls acarbose replaced the flavenoids. Mixtures without enzyme, flavonoids or acarbose served as blanks. The reaction mixtures were incubated at 25° C. for 5 min, after which the reaction was stopped by boiling for 2 min. Absorbance of the resulting p-nitrophenol (pNP) were spectrophotometry determined at 405 nm and was considered directly proportional to the activity of the enzyme. Glucosidase activity inhibition was determined as percentage of control as follows:

$$\% Glucosidase inhibition = 100 \% - \% activity of test as percentage of control \\ \% Activity of test = \frac{corrected A 405 of test \times 100 \%}{A 405 of controls}$$

In order to eliminate background readings, the absorbance of the flavenoid without substrate and enzyme was subtracted from absorbance of the flavonoid and substrate mixture as follows: Corrected A₄₀₅ test samples=A₄₀₅ flavonoid and substrate—A₄₀₅ flavonoid alone (background).

The activity in controls (with a-glucosidase but without inhibitor) was considered to be 100%. Concentrations of extracts resulting in 50% inhibition of enzyme activity (ICso values) were determined graphically. Different flavonoids were compared on the basis of their ICso values estimated from the dose response curves.

| Flavonoid Compound (inhibition | |
|---|---|
| of yeast alpha-glucosidase) | IC₅₀ (μM) |
| Myricetin | 3.6 |
| Dihydromyricetin | 4.0 |
| Quercetin | 8.0 |
| Luteolin | 35 |
| Baicalein | 58 |
| Kaempferol | 72 |
| Apigenin | 90 |
| Phloretin | 96 |
| Chrysin | <20% * 200 uM^{a} |
| Acarbose (positive control) | 562 |

Myricetin, quercetin, and dihydromyricetin used in the compositions, were affirmed to be powerful enzyme inhibitors, thus supporting their use.

Similar studies were conducted to determine the inhibition of the above compounds on alpha amylase and it was established that quercetin dihydromyricetin and myricetin also are effective at inhibiting amylase.

Individually, quercetin, dihydromyricetin and myricetin inhibit amylase as well as alpha-glucosidases. In physiological context, this translates to an inhibition of pancreatic amylase, digestive amylase, and enzymes sucrase-isomaltase and maltase-glycoamylase at the brush border of the small intestines.

1-28. (canceled)

29. A process for manufacturing a formulation for modulating a glycemic response comprising:
a. mixing at least two different phenylpropanoids and a first cyclodextrin;
b. adding water to the mix of at least two different phenylpropanoids and the first cyclodextrin to form a paste;
c. kneading the paste with shear force;
d. drying the paste;
e. grinding the dried paste to a powder; and
f. adding an iminosugar, a monosaccharide-based enzyme inhibitor and a second cyclodextrin to the powder to constitute the formulation,
wherein the powder comprises the phenylpropanoids encapsulated in the first cyclodextrin.

30. The process according to claim 29, wherein the first and second cyclodextrin are different.

31. The process according to claim 29, wherein the first and second cyclodextrin are selected from the group consisting of alpha cyclodextrin, beta cyclodextrin and gamma cyclodextrin.

32. The process according to claim 29, wherein the first cyclodextrin comprises gamma cyclodextrin

33. The process according to claim 29, wherein the at least two different phenylpropanoids comprises a flavonoid, a chalcone, or any combination thereof.

34. The process according to claim 29, wherein the at least two different phenylpropanoids are selected from the group consisting of one or more flavonoid, at least chalcone, and any combination thereof.

35. The process according to claim 33, wherein the flavonoid comprises: quercetin, myricetin, dihydromyricetin, luteolin, baicalein, apigenin, kaempferol, or any combination thereof.

36. The process according to claim 33, wherein the at least one chalcone comprises: dihydrochalcone glycoside, phlorizin or phloretin.

37. The process according to claim 29, wherein the first cyclodextrin is gamma cyclodextrin; the at least twodifferent phenylpropanoids are quercetin, myricetin, and phlorizin; the iminosugar comprises 1-deoxynojirimycin; the monosaccharide-based enzyme inhibitor is arabinose; and
the second cyclodextrin is alpha cyclodextrin.

38. The process according to claim 37, wherein the gamma cyclodextrin:quercetin:myricetin:phlorizin:1-deoxynojirimycin:arabinose:alpha cyclodextrin is manufactured in a ratio of 40:9:9:5:5:10:22 or a ratio of

39. The process according to claim 29, wherein the at least two different phenylpropanoids comprise dihydromyricetin, and dihydrochalcone glycoside-phlorizin.

40. The process according to claim 29, wherein the first cyclodextrin is gamma cyclodextrin; the at least two different phenylpropanoids are dihydromyricetin, phlorizin; the iminosugar is 1-deoxynojirimycin; the monosaccharide-based enzyme inhibitor is arabinose; and the second cyclodextrin is alpha cyclodextrin.

41. The process according to claim 40, wherein the ratio of gamma cyclodextrin:dihydromyricetin:phlorizin:1-deoxynojirimycin:arabinose:alpha cyclodextrin is 5:2:1:3:1:30 to 25:6:6:10:10:90.

42. The process according to claim 29, further comprising adding a flavour or colour varying additive.

43.-56 (canceled)

57. A formulation for modulating a glycemic response comprising:
a. at least two different phenylpropanoids encapsulated in a first cyclodextrin;
b. an iminosugar;
c. a monosaccharide-based enzyme inhibitor and
d. a second cyclodextrin, wherein the first cyclodextrin is different from the first cyclodextrin.

58. The formulation according to claim 57, wherein the first and second cyclodextrin are selected from the group consisting of alpha cyclodextrin, beta cyclodextrin and gamma cyclodextrin and the first cyclodextrin comprises gamma cycodextrin.

59. The formulation according to claim 57, wherein the at least two different phenylpropanoids are selected from the group consisting flavonoid, chalcone, and any combination thereof.

60. The formulation according to claim 59, wherein the flavonoid comprises:
quercetin, myricetin, dihydromyricetin, luteolin, baicalein, apigenin, kaempferol, or any combination thereof.

61. The formulation according to claim 59, wherein the at least one chalcone comprises: dihydrochalcone glycoside, phlorizin or phloretin.

62. The formulation according to claim 57, wherein the first cyclodextrin is gamma cyclodextrin; the at least two different phenylpropanoids are quercetin, myricetin, phlorizin; the iminosugar comprises 1-deoxynojirimycin; the monosaccharide-based enzyme inhibitor is arabinose; and the second cyclodextrin is alpha cyclodextrin.

63. The formulation according to claim 62, wherein the ratio of gamma cyclodextrin:quercetin:myricetin:phlorizin:1-deoxynojirimycin:arabinose:alpha cyclodextrin is 40:9:9:5:5:10:22 or 35:9:9:4:5:37:1.

64. The formulation according to claim 57, wherein the at least two different phenylpropanoids comprise dihydromyricetin, and dihydrochalcone glycoside-phlorizin.

65. The formulation according to claim 57, wherein the first cyclodextrin is gamma cyclodextrin; the at least two different phenylpropanoids are dihydromyricetin, phlorizin; the iminosugar comprises 1-deoxynojirimycin; the monosaccharide-based enzyme inhibitor is arabinose; and the second cyclodextrin is alpha cyclodextrin.

66. The formulation according to claim 65, wherein the ratio of gamma cyclodextrin:dihydromyricetin:phlorizin:1-deoxynojirimycin:arabinose:alpha cyclodextrin is 5:2:1:3:1:30 to 25:6:6:10:10:90.

66. The formulation according to claim 57, further comprising a flavour or colour varying additive.

67. A foodstuff additive including the formulation according to claim 57.

68. A method for treating or preventing diabetes comprising administering to an individual an amount of a formulation according to claim 57 to reduce the glycemic response of the individual.
